(19) United States
(12) Patent Application Publication
(10) Pub. No.: US 2014/0371097 A1
(43) Pub. Date: Dec. 18, 2014

(54) NON-INVASIVE METHODS FOR ASSESSING OOCYTE QUALITY FOR IN VITRO FERTILIZATION

(71) Applicant: THE REGENTS OF THE UNIVERSITY OF CALIFORNIA, Oakland, CA (US)

(72) Inventors: Marco Conti, San Francisco, CA (US); Alberuni Musa Zamah, San Francisco, CA (US); Jing Chen, San Francisco, CA (US)

(21) Appl. No.: 14/366,230

(22) PCT Filed: Dec. 21, 2012
(86) PCT No.: PCT/US2012/071417

§ 371 Date: Jun. 17, 2014

Related U.S. Application Data
(60) Provisional application No. 61/578,666, filed on Dec. 21, 2011.

(51) Int. Cl. G01N 33/68; C12Q 1/68; G01N 33/74

(52) U.S. Cl. CPC G01N 33/689; G01N 33/74; C12Q 1/6804; G01N 33/6869; G01N 2333/5418; G01N 2333/485; G01N 2333/495; G01N 2333/50
USPC 506/9; 435/6.11; 435/6.12; 435/7.1; 506/18

(57) ABSTRACT

The present invention provides methods of selecting oocytes for in vitro fertilization comprising determining the level of translation products in medium in which the oocyte is incubate.

### CROSS-REFERENCES TO RELATED APPLICATIONS

This application claims benefit of U.S. Provisional Application No. 61/578,666, filed Dec. 21, 2011, the disclosure of which is hereby incorporated by reference in its entirely for all purposes.

### STATEMENT AS TO RIGHTS TO INVENTIONS MADE UNDER FEDERALLY SPONSORED RESEARCH AND DEVELOPMENT

This invention was made with Government support under National Institutes of Health (NIH) Grant Nos. HD052909-01 (29187) and GM080527-01 (29183). The Government has certain rights to this invention.

### BACKGROUND OF THE INVENTION

In current in vitro fertilization (IVF) methods, multiple embryos are routinely transferred in view of the inability to recognize those embryos that will produce a viable pregnancy. This is a widely accepted practice, particularly in older reproductive-age women, even though it is well established that adverse effects are associated with multiple embryo transfer resulting in multiple gestation [1]. This includes increased maternal complications (e.g., pre-eclampsia) and increased morbidities and mortality of neonates (e.g., pre-term delivery, neuro-developmental disorders).

Oocytes traverse two critical maturation steps around the periovulatory period. The nucleus undergoes changes (nuclear maturation) required for the production of gametes with the correct ploidity and genetic information ([2], [3]). At the same time, additional poorly defined changes collectively called “cytoplasmic maturation”, including organelle relocation within the cytoplasm of the egg, must take place in the oocyte ([4], [5]). Both nuclear and cytoplasmic maturation are essential for the oocyte to acquire the competence to develop into an embryo. They must they take place prior to fertilization and also must occur in synchrony [6]. As transcription is silent in the fully grown oocyte, developmental competence relies largely on a post-transcriptional program of selective recruitment of maternal mRNA to the oocyte polysomes for protein synthesis [3]. Although the importance of translational regulation for embryonic development is undisputed, very little is known about this program at the molecular level.

Currently available non-invasive screening methods to identify oocytes that are likely to produce embryos that will be viable are based on morphological observations and embryo scoring or monitoring the rate of cleavage. These assessments are not quantitative, are difficult to standardize, and are subjective. Thus, there is a need to develop new strategies to assess the quality of retrieved oocytes and of embryos that are to be transferred. This invention provides a method to efficiently and reproducibly assess oocyte quality in terms of the ability of the oocyte to be fertilized and develop as a viable embryo.

### BRIEF SUMMARY OF THE INVENTION

This invention relates, in part, to the discovery that in many cases of infertility and IVF failure, complex programs that results in proper translation of a competent RNA repertoire are not correctly executed. Accordingly, the current invention provides methods of evaluating oocyte competency by determining the translation profile of oocytes. The method can be performed using oocytes from any mammal.

In one aspect, the invention provides a method of identifying an oocyte that is suitable for use in in vitro fertilization, the method comprising: culturing a candidate oocyte in an oocyte culture medium; obtaining a sample of the medium in which the oocyte has been cultured for a defined period of time, e.g., 4 to 12 hours; analyzing a profile of proteins secreted in to the media by the oocyte or embryo, that reflects the translational program. The analysis comprises determining the amounts of multiple proteins selected from a group of secreted proteins consisting of, but not limited to, BMP15, IL-7, FGF1, TGFβ2, TGFβ3, PTHPIP, Def41b, BMP5, Gremlin1, Rspo2, EGF, and CSF1 in the culture medium; and comparing the amount of the least one protein to the amount in the spent medium from a competent oocyte. In some embodiments, the oocyte is fertilized.

These secreted targets are identified on the basis of measurements of translation at varying states of oocyte maturation and in a model of impaired oocyte competence (see Table 1). In some embodiments, the amounts of at least two proteins selected from BMP15, IL-7, FGF1, TGFβ2, TGFβ3, PTHPIP, Def41b, BMP5, Gremlin1, Rspo2, EGF, and CSF1 are determined. In some embodiments, the amounts of at least three proteins selected from BMP15, IL-7, FGF1, TGFβ2, TGFβ3, PTHPIP, Def41b, BMP5, Gremlin1, Rspo2, EGF, and CSF1 are determined. In some embodiments, the amounts of at least four proteins selected from BMP15, IL-7, FGF1, TGFβ2, TGFβ3, PTHPIP, Def41b, BMP5, Gremlin1, Rspo2, EGF, and CSF1 are determined. In some embodiments, the amounts of at least five proteins selected from BMP15, IL-7, FGF1, TGFβ2, TGFβ3, PTHPIP, Def41b, BMP5, Gremlin1, Rspo2, EGF, and CSF1 are determined. In some embodiments, the amounts of at least six proteins selected from BMP15, IL-7, FGF1, TGFβ2, TGFβ3, PTHPIP, Def42b, BMP5, Gremlin1, Rspo2, EGF, and CSF1 are determined. In some embodiments, the levels of more than one, e.g., 2, 3, 4, 5, 6, 7, 8, 9, 10, 11, or 12, proteins selected from BMP15, IL-7, FGF1, TGFβ2, TGFβ3, PTHPIP, Def41b, BMP5, Gremlin1, Rspo2, EGF, and CSF1 are determined. In some embodiments, the levels of BMP15, IL-7, FGF1, TGFβ2, TGFβ3, PTHPIP, Def41b, BMP5, Gremlin1, Rspo2, EGF, and CSF1 are determined.

In a further aspect, the invention also provides a method to detect the levels of a plurality of oocyte secreted proteins in a single assay. In typical embodiments, the kit comprises reagents for detecting at least 2, 3, 4, 5, 6, 7, 8, 9, 10, 11, or 12 more proteins selected from, but limited to, BMP15, IL-7, FGF1, TGFβ2, TGFβ3, PTHPIP, Def41b, BMP5, Gremlin1, Rspo2, EGF, and CSF1. In some embodiments, the kit further comprises reagents for detecting at least one protein selected from the groups of secreted proteins described above. In some embodiments, the kit comprises reagents for a DNA barcode assay, e.g., magnetic beads to which a primary antibody to the protein to be detected is attached and a secondary antibody that binds to the protein, attached to a DNA barcode sequence.

Other objects, features, and advantages of the present invention will be apparent to one of skill in the art from the following detailed description and figures.

### BRIEF DESCRIPTION OF THE DRAWINGS

FIG. 1. a, b. Schematic of DNA barcode immunoassay.

FIG. 2. (a). Example of a standard curve for quantifying a barcode qPCR reaction. (b) Increased sensitivity of detection of DNA barcode immunoassay versus conventional Enzyme-linked ImmunoSorbent Assay (ELISA)

FIG. 3. Translation of mRNA coding for secreted proteins in oocytes from AREG knockout mice compared to normal mice. The two groups compared are oocytes at the stage of development (these are oocytes ready to be fertilized, also called eggs; this is the stage retrieved during IVF). The competent oocytes come from wild type mice, whereas the incompetent oocytes are from a KO model (AREG KO). These mice have reduced fertility and about 50% of the oocytes do not fertilize when tested in an in vitro fertilization protocol (IVF protocol).

FIG. 4. Measurement of Secreted AREG into IVF conditioned media from human cumulus-oocyte (COC) complexes using DNA barcode immunoassay.

### DETAILED DESCRIPTION OF THE INVENTION

As used herein, the following terms have the meanings ascribed to them unless specified otherwise.

As used herein, the term “oocyte maturation” refers to biochemical events that prepare an oocyte for fertilization. Such processes may include, but are not limited to, the completion of meiosis II. Oocyte cytoplasmic maturation events may include, but are not limited to, mRNA translation and protein accumulation to achieve competence for fertilization. In the present invention, oocytes that are analyzed are arrested at metaphase of meiosis II.

The term “competent” with respect to an oocyte refers to the ability of the oocyte to be fertilized and produce a viable embryo.

In the context of this invention, “an amount of a protein that is associated with competence” refers to the amount of secreted protein present in a spent media following a defined period of incubation of an oocyte in the media where the amount is that secreted by a known competent oocyte. In some embodiments, the oocyte may be a fertilized oocyte (i.e., embryo).

A “patient” in the context of this invention refers to any mammal.

A “translation profile” in the context of this invention refers to the amount of proteins of interest in a secreted into a medium by an oocyte or embryo. In some embodiments the amounts of at least 7 proteins are determined in a translation profile.

The term “TGFβ2” in the context of the present invention refers to transforming growth factor beta 2 protein, which is a member of the transforming growth factor beta (TGFB) family of growth factors. An example of a human TGFβ2 protein sequence is available under accession number UniProtKB/Swiss-Prot P61812. An example of a human nucleic acid sequence encoding TGFβ2 is available under accession number NM_{—}001135599. Human TGFβ2 is localized to chromosome 1q41. TGFβ2 refers to allelic variants that are encoded by the gene at the TGFβ2 chromosomal locus.

The term “TGFβ3” in the context of the present invention refers to transforming growth factor beta 3 protein, which is a member of the transforming growth factor beta (TGFβ) family of growth factors. An example of a human TGFβ3 protein sequence is available under accession number UniProtKB/Swiss-Prot P10600. An example of a human DNA sequence encoding TGFB3 is available under accession number NM_{—}003239. Human TGFβ3 is localized to chromosome 14q24. “TGFBβ” also encompasses allelic variants of the exemplary references sequence that are encoded by a gene at the TGFβ3 chromosomal locus.

The term “EGF” in the context of the present invention refers to epidermal growth factor protein protein. An example of a human EGF protein sequence is available under accession number UniProtKB/Swiss-Prot P01133. An example of a human nucleic acid sequence encoding EGF is available under accession number NM_{—}001178130. Human EGF is localized to chromosome 4q25. “EGF” also encompasses allelic variants of the exemplary references sequence that are encoded by a gene at the EGF chromosomal locus.

The term “Defb41” is mouse beta-defensin 41 precursor, but as used herein also encompasses the human ortholog beta-defensin 110 isoform b. An example of a human Defb41 protein sequence is available under accession number Q30KQ9. An example of a human nucleic acid sequence encoding Defb41 is available under accession number NM_{—}001037497. Human Defb41 is localized to chromosome 6p12.3 “Defb41” also encompasses allelic variants of the exemplary references sequence that are encoded by a gene at the Defb41 chromosomal locus.

The term “PTHPIP” or “PTHrP” in the context of the present invention refers to parathyroid hormone like hormone (Pthlh). In the mouse, the gene is referred to as parathyroid hormone like peptide. An example of a human PTHPIP protein sequence is available under accession number UniProtKB: P12272. An example of a mouse sequence is NM_{—}008970.3. Examples of human nucleic acid sequences encoding PTHPIP are available under accession numbers NM_{—}002820.2, NM_{—}198964.1, NM_{—}198965.1, and NM_{—}198966.1. Human PTHPIP is localized to Entrez Gene cytogenetic band: 12p12.1-p11.2. “PTHPIP” also encompasses allelic variants of the exemplary references sequence that are encoded by a gene at the PTHPIP chromosomal locus.

The term “GDF9” in the context of the present invention refers to growth/differentiation factor 9 protein. An example of a human GDF9 protein sequence is available under accession number UniProtKB/Swiss-Prot O60383. An example of a human nucleic acid sequence encoding GDF9 is available under accession number NM_{—}005260. Human GDF9 is localized to cytogenetic band 5q31.1. “GDF9” also encompasses allelic variants of the exemplary references sequence that are encoded by a gene at the GDF9 chromosomal locus.

The term “BMP5” in the context of the present invention refers to bone morphogenetic protein 5 protein. An example of a human BMP5 protein sequence is available under accession number UniProtKB/Swiss-Prot P22003. An example of a human nucleic acid sequence encoding BMP5 is available under accession number NM_{—}021073. Human BMP5 is localized to cytogenetic band 6p12.1. “BMP5” also encompasses allelic variants of the exemplary references sequence that are encoded by a gene at the BMP5 chromosomal locus.

The term “BMP15” in the context of the present invention refers to bone morphogenetic protein 15 protein. An example of a human GDF9 protein sequence is available under accession number UniProtKB/Swiss-Prot O95972. An example of a human nucleic acid sequence encoding BMP15 is available under accession number NM_{—}005448. Human BMP15 is localized to cytogenetic band Xp11.2. “BMP15” also encompasses allelic variants of the exemplary references sequence that are encoded by a gene at the BMP15 chromosomal locus.

The term “IL-7” or “IL7” in the context of the present invention refers to interleukin-7 protein. An example of a human IL-7 protein sequence is available under accession number UniProtKB/Swiss-Prot P13232. An example of a human nucleic acid sequence encoding IL-7 is available under accession number NM_{—}000880. Human IL-7 is localized to Entrez Gene cytogenetic band 8q12-q13. “IL-7” also encompasses allelic variants of the exemplary references sequence that are encoded by a gene at the IL-7 chromosomal locus.

The term “CSF1” in the context of the present invention refers to macrophage colony-stimulating factor protein. An example of a human CSF1 protein sequence is available under accession number UniProtKB/Swiss-Prot P09603. An example of a human nucleic acid sequence encoding CSF1 is available under accession number NM_{—}000757. Human CSF1 is localized to Esembl cytogenetic band 1p13.3. “CSF1” also encompasses allelic variants of the exemplary references sequence that are encoded by a gene at the CSF1 chromosomal locus.

The term “FGF1” in the context of the present invention refers to fibroblast growth factor 1 protein. An example of a human FGF1 protein sequence is available under accession number UniProtKB/Swiss-Prot P05230. An example of a human nucleic acid sequence encoding FGF1 is available under accession number NM_{—}000800. Human FGF1 is localized to cytogenetic band 5q31. “FGF1” also encompasses allelic variants of the exemplary references sequence that are encoded by a gene at the FGF1 chromosomal locus.

The term “Gremlin1” or “GREM1” in the context of the present invention refers to Gremlin1 protein. Alternative names include cell proliferation-inducing gene 2 protein, cysteine knot superfamily 1, BMP antagonist 1, DAN domain family member 2, down-regulated in Mos-transformed cells protein, increased in high glucose protein 2, and IHG-2. An example of a human Gremlin1 protein sequence is available under accession number UniProtKB/Swiss-Prot O60565. An example of a human nucleic acid sequence encoding Gremlin1 is available under accession numbers NM_{—}0010091323.1 and NM_{—}01337206. Human Gremlin1 is localized to cytogenetic band 15q13.3. “Gremlin1” also encompasses allelic variants of the exemplary references sequence that are encoded by a gene at the Gremlin1 chromosomal locus.

The term “Rspo2” in the context of the present invention refers to R-spondin-2 or roof place specific spondin-2 protein. An example of a human Rspo2 protein sequence is available under accession number UniProtKB/Swiss-Prot Q6UXX9. An example of a human nucleic acid sequence encoding Rspo2 is available under accession number NM_{—}0178565.4. Human Rspo2 is localized to cytogenetic band 8q23.1. “Rspo2” also encompasses allelic variants of the exemplary references sequence that are encoded by a gene at the Rspo2 chromosomal locus.

The term “antibody” refers to an immunoglobulin which specifically binds to an antigen. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained. Antibodies to the proteins of interest are commercially available or can be obtained using known techniques as noted above.

As used here “barcode”, “biochemical barcode”, “biobarcode”, “barcode oligonucleotide”, or “barcode DNA” are used interchangeably.

The term “particle” or “bead” refers to a small surface, typically composed of a metal, silica, silicon-oxide, or polystyrene to which a substrate may be attached. A “particle” can be any shape, such as spherical or rod-shaped.

### Method of Identifying Competent Oocytes

The present invention relates to methods of identifying oocytes that are competent. The methods comprise evaluating oocytes for secreted proteins that are indicative of oocyte competence. The methods and compositions described herein can be used to evaluate oocytes from any mammal. In some embodiments, the oocytes are from humans. Other mammalian oocytes that can be assessed for secreted proteins include, but are not limited to, bovine, equine, ovine, caprine, porcine, feline, canine, or non-human primate, such as simian, oocytes.

### Oocyte Retrieval and Culture

Oocytes are retrieved using known procedures. In some embodiments, the oocytes are from a human. In other embodiments, oocytes are retrieved from a non-human mammal such as a cow, horse, pig, or sheep. Oocyte retrieval and culture techniques using human oocytes are used here as illustrative examples. One of skill understands that oocytes can be retrieved from non-human mammals using known techniques and similarly cultured.

For purposes of illustration and not limitation, in some embodiments, oocytes are retrieved from human patients undergoing controlled ovarian stimulation during treatment for in vitro fertilization (IVF). In a typical IVF cycle, a hCG bolus is given to complete the final stages of oocyte maturation. Oocyte retrieval is typically performed using ultrasound-guided needle aspiration of the ovarian follicles. Cumulus-oocyte complexes, which are the oocytes and cumulus cells that surround the oocytes, are then isolated from the ovarian follicular fluid.

Following isolation, individual cumulus-oocyte complexes are cultured in defined medium, following which the medium is collected. The cumulus-oocyte complexes are incubated for at least 3 hours and preferably at least 5 or 6 hours, and in some embodiments at least 7, 8, 9, 10, 11, or 12 hours, in defined medium, preferably at 37° C. At the end of the incubation, the cumulus-oocyte complex is transferred to fresh medium and the incubation medium is collected for analysis.

In embodiments in which the oocyte is fertilized, fertilizing the oocytes to obtain an embryo is performed by standard in vitro fertilization techniques. The oocytes are either directly incubated with spermatozoa for IVF or are stripped of the cumulus cells, scored for the stage of maturation, and mature MII oocytes are fertilized by intracytoplasmic sperm injection (ICSI). The oocytes are then incubated, typically for about 14-20 hours, and then assessed by microscopy for normal fertilization, as defined by the presence of two pronuclei within the newly fertilized oocyte. At the end of this second incubation spent media are collected, (embryo spent medium). Embryos may be incubated for an additional time period (e.g., a total of 3-5 days) and scored for morphological and/or genetic measures of embryo quality. In the present invention, an assay of secreted proteins present in the culture medium sample (embryo spent media) is also employed to select embryos that will be transferred.

Any number of defined media are used for oocyte or embryo incubation. For example, commercially available mammalian cell culture media may be used. Examples of media that are suitable for incubating oocytes or embryos include formulations using minimal media such as MEMα, Ham's F12, DMEM, with supplements, and the like. In typical embodiments, the oocytes or embryos are cultured in commercially available in vitro fertilization medium. As understood in the art, when spent medium is collected to be evaluated for the presence of secreted proteins to identify competent oocytes or to select embryos, serum or other complex mixtures of proteins are not included in the culture medium. Examples of commercially available media include Vitrolife G-1 medium (Vitrolife Göteborg, Sweden). In some embodiments, the media are supplemented with a serum substitute, e.g., serum substitute supplement, Irvine Scientific, or HSA (human serum albumin).

### Assay of Oocyte and Embryo Secretory Products

In the present invention, oocyte and embryo quality determination is based on their secretion of proteins being synthesized during oocyte maturation and early embryo development. Examples of secretory products affected by oocyte quality include EGF, TGFβ2, TGFβ3, PTHPIP, IL-7, Defb41, Rspo2, CSF1, and BMP5. There are also secreted proteins where the level of protein in the medium is not affected, which can be used as controls. Secretory products not affected and that can be used as control reference levels of secreted proteins include Gremlin1, BMP15 and FGF1.

Table 1 shows representative genes coding for proteins secreted by the oocytes and their pattern of translation during maturation, as well as the changes that can occur in oocytes or embryos with compromised developmental competence. The list of genes expressed by the oocyte was derived from microarray data. Changes in translation in the mRNA during maturation were assessed by measuring the level of corresponding mRNAs in the polysome fraction using array hybridization or qPCR. Changes in oocytes with compromised quality were derived from the analysis of the Areg knockout mouse or from available human data. As understood in the art, there may be alterations to these patterns. However, in the embodiments of the invention, evaluation of a secreted protein panel comprising one or more of BMP15, IL7, FGF1, TGFβ3, TGFβ2, PTHPIP, Def41b, BMP5, Gremlin1, Rspo2, EGF, or CSF1, preferably two, three, four, five, six, seven, eight, or nine or more of these secreted proteins in comparison to normal allows one of skill to determine the likelihood of an oocyte having comprised competence. In some embodiments, evaluation of secreted proteins to assess oocyte competence comprises determining the amount of secretion of at least one protein selected from IL-7; CSF1, PTHPIP, TGFβ2, and EGF. In some embodiments, evaluation of secreted proteins to assess oocyte competence comprises determining the amounts of secretion of at least two proteins selected from IL-7; CSF1, PTHPIP, TGFβ2, and EGF. In some embodiments, evaluation of secreted proteins to assess oocyte competence comprises determining the amounts of secretion of at least three proteins selected front IL-7; CSF1, PTHPIP, TGFβ2, and EGF. In some embodiments, evaluation of secreted proteins to assess oocyte competence comprises determining the amounts of secretion of at least four proteins selected from IL-7; CSF1, PTHPIP, TGFβ2, and EGF. In some embodiments, evaluation of secreted proteins to assess oocyte competence comprises determining the amounts of secretion of at least five proteins, wherein the at least five proteins are IL-7; CSF1, PTHPIP, TGFβ2, and EGF. In some embodiments, evaluation of secreted proteins to assess oocyte competence comprises determining the amounts of secretion of at least four proteins, wherein the at least four proteins are IL-7; CSF1, PTHPIP and EGF.

TABLE 1
Genes of secreted proteins and mRNA changes in oocytes

| | Changes in | Changes in |
|---|---|---|
| | translation | model of |
| Secreted | during | compromised |
| protein | maturation | competence |
| BMP15 | ↓ | =↑ |
| IL7 | ↑ | ↓ |
| FGF1 | = | = |
| TGFβ3 | ↓ | ↓ |
| TGFβ2 | ↓ | ↓ |
| PTHPIP | ↑ | ↓ |
| Def41b | ↑ | ↓ |
| BMP5 | ↑ | = |
| Gremlin1 | =↑ | = |
| Rspo2 | =↑ | =↓ |
| EGF | ↑ | =↑ |
| CSF1 | ↑ | =↓ |

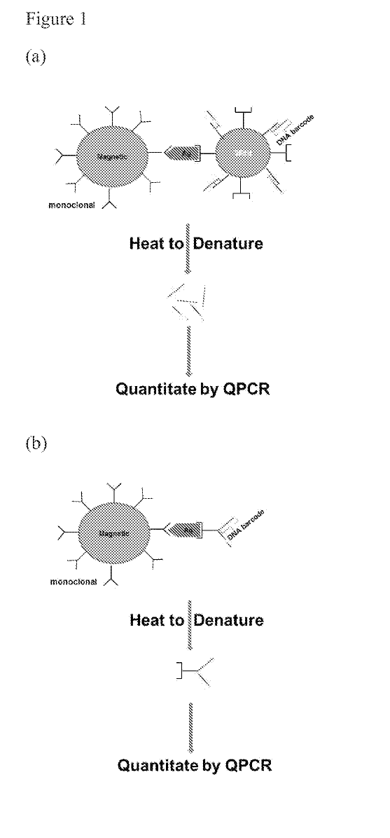

Although levels of secreted proteins may be measured individually, in typical assays, more than one secreted protein is measured simultaneously using a multiplex assay. In a preferred embodiment, the assay of oocyte- or embryo-secreted factors in spent media from oocytes or embryos takes advantage of a method termed “bio-barcode” amplification, which allows for detecting amount of target proteins with attomolar sensitivity. Bio-barcode assays are known. See, e.g., U.S. Patent Application Publication No. 20100081134, WO/2006/078289, WO/2006/125050, and WO2007/084192, each of which is incorporated by references. An illustrative schematic providing an example of this type of assay is provided in FIG. 1.

Other highly sensitive assay methods may also be employed to analyze proteins secreted by oocytes into culture medium. For example, cumulus cells may be transformed with a luciferase or a fluorescent reporter to detect growth factors, e.g., TGFβ2, TGFβ3, EGF, present in oocyte culture medium using an assay analogous to the TopFlash assay used to measure Wnt signals (see, e.g., reference [7]).

Other examples of techniques that can be used to measure protein secretion into culture medium include magnetic nanotag sensing, (see, e.g., reference [8]).

In this section, a bio-barcode assay is described to illustrate, but not to limit, the invention. In performing a bio-barcode assay, target capture molecules and barcode DNA strands are conjugated, and either linked or not linked to microparticles, e.g., magnetic or silica bead. In some embodiments, magnetic beads are conjugated to a primary antibody directed against a protein to be analyzed. Beads, e.g., silica beads, are conjugated to a secondary antibody that binds to the protein, and to an oligonucleotide complementary to a bar code DNA. After extensive washing, the coupled silica beads are incubated in hybridization conditions with the DNA barcode. Barcode DNA sequences are well known in the art (see, e.g., U.S. Patent Application Publication No. 20100081134, WO/2006/078289, WO/2006/125050, and WO2007/084192). Any barcode sequence may be used. Primary antibodies are available commercially or may be generated using well known techniques. A secondary antibody may bind to the protein directly or to the primary antibody. For example, a primary antibody may additionally be conjugated to another molecule that provides for amplification of a signal generated by binding of the protein to a primary antibody.

After hybridization, the conjugated antibody/DNA barcode beads and the conjugated primary antibody magnetic beads are washed and may be stored.

As noted above, antibodies to the proteins of interest are commercially available or can be prepared using known methodology.

### Target Protein Capture

Spent media from oocytes and embryos are incubated with a defined amount of primary antibody-conjugated beads and the secondary antibody/barcode. A standard curve of known concentration of the protein of interest is also typically prepared for quantification. Following incubation, complexes comprising the bead having the primary antibody conjugated to it and the secondary antibody/barcode are formed when the protein of interest is present. In embodiments, in which a magnetic bead is used for the conjugation of the primary antibody, a magnetic separator may be used to obtain the complexes with the magnetic particles along with captured proteins and the DNA barcode-coupled secondary antibody. Supernatants are removed and the magnetic bead pellet washed. After the last wash, complexes are resuspended and incubated to release the DNA barcode from the complex.

### Detection of Released Barcode DNA

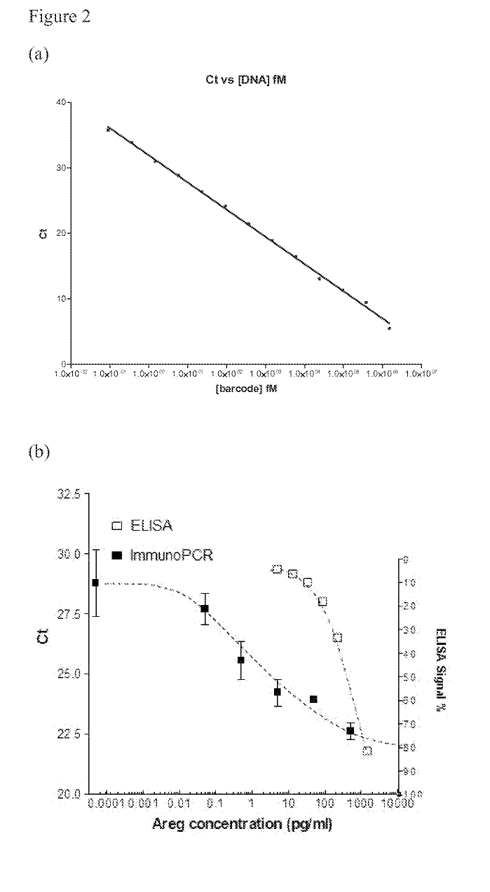

Aliquots of supernatant released from the complexes are subjected to standard qPCR using pairs of primers specific for each barcode DNA. An example of a standard curve is provided in FIGS. 2a and b. The amount of protein secreted by the oocytes or embryos into the spent medium is then computed by comparing the Cₜ values from the standard curve to the Cₜ values from the samples being analyzed. These protein levels are then used in combination to assess overall oocyte and embryo health.

The methods of the invention comprise determining the amounts of at least one, and preferably more than one of the secreted proteins including, but not limited to, BMP15, IL-7, FGF1, TGFβ2, TGFβ3, PTHPIP, Def41b, BMP5, Gremlin1, Rspo2, EGF, and CSF1 in the spent medium. For example, oocytes that are not competent may secrete very little PTHPIP, Defb41, TGFβ3 in spent media compared to competent oocytes and about 50% less TGFβ2 and EGF. Oocytes that are selected for in vitro fertilization preferably show an amount in spent media for at least one, e.g., 1, 2, 3, 4, 5, 6, 7, 8, 9, 10, 11, or 12 proteins that is associated with competence. In some embodiments, oocytes are selected for in vitro fertilization that have amounts of secreted proteins in spent media for nine of the proteins, described above, that are associated with competence. In some embodiments, oocytes are selected for in vitro fertilization that have amounts of secreted proteins in spent media for seven of the proteins that are associated with competence. In some embodiments, oocytes are selected for in vitro fertilization that have amounts of secreted proteins in spent media for five of the proteins that are associated with competence. For example, in some embodiments, oocytes that are selected for in vitro fertilization are selected for in vitro fertilization that secrete levels of Defb41, PTHPIP, TGFβ2, TGFβ3, IL-7, CSF1, and EGF that are associated with competence. In some embodiments, oocytes are selected for in vitro fertilization that secrete levels of IL-7, PTHPIP, TGFβ2, CSF1, and EGF that are associated with competence. In some embodiments, oocytes are selected for in vitro fertilization that secrete levels of IL-7, CSF1, PTHPIP, and EGF that are associated with competence. In some embodiments, the levels of IL-7, CSF1, PTHPIP, TGFβ2, and EGF associated with competence reflect the pattern observed in Table 1.

### Controls

A standard curve is typically also run as a control for determining the amounts of the proteins of interest in incubation media. In some embodiments, the values for the amounts of protein obtained in an analysis are compared to reference values obtained from analyzing protein levels in secreted media from competent oocytes.

In some embodiments, the values for the amounts of protein obtained in an analysis are compared to reference values obtained from analyzing protein levels in secreted media from oocytes that are not competent.

Those skilled in the art are familiar with various ways of using reference values. The reference values may be determined using a concurrent analysis of competent or not competent oocytes, but in typical embodiments, are from known values. Thus, in some embodiments, the value representing the amount of secreted protein in the culture medium is compared to one or more reference values, and optionally correlated to oocyte competence.

The information obtained from the secreted protein analysis may be stored in a computer readable form. Such a computer system typically comprises major subsystems such as a central processor, a system memory (typically RAM), an input/output (I/O) controller, an external device such as a display screen via a display adapter, serial ports, a keyboard, a fixed disk drive via a storage interface, and, for example, a CD-ROM (or DVD-ROM) device operative to receive a CD-ROM. Many other devices can be connected, such as a network interface connected via a serial port.

The computer system also be linked to a network, comprising a plurality of computing devices linked via a data link, such as an Ethernet cable (coax or 10 BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired front an assay of the invention.

The computer system can comprise code for interpreting the results of a study evaluating the amount of secreted protein in an analysis. Thus in an exemplary embodiment, the secreted protein analysis results are provided to a computer where a central processor executes a computer program for determining the likelihood of having a competent oocyte.

### Kits

The invention additionally provides compositions and kits comprising sensitive assays, e.g., bio-barcode assays to detect the levels of secreted proteins in oocyte or embryo incubation media. In some embodiments, the kit comprises reagents to detect at least two, often at least three, four, five, six, seven, eight, nine, ten, eleven secreted proteins selected from a group consisting of BMP15, IL-7, FGF1, TGFβ2, TGFβ3, PTHPIP, Def41b, BMP5, Gremlin1, Rspo2, EGF, and CSF1, or combinations thereof. In some embodiments, the kit comprises reagents to detect secreted BMP15, IL-7, FGF1, TGFβ2, TGFβ3, PTHPIP, Def41b, BMP5, Gremlin1, Rspo2, EGF, and CSF1. In some embodiments, the kit comprises reagents to detect secreted IL-7, EGF, TGFβ2, TGFβ3, PTHPIP, CSF1, BMP5, Rspo@, or Defb41, or combinations thereof comprising at least two, three, four, five, six, seven, or eight of the proteins. In some embodiments, the kit further comprises reagents to detect secretion of one or more of the proteins BMP15, FGF1, and Gremlin1. In some embodiments, the kit comprises reagents to detect secreted IL-7; CSF1, PTHPIP, TGFβ2 and EGF. In some embodiments, the kit comprises reagents to detect secreted IL-7; CSF1, PTHPIP, and EGF. These are examples of the secreted proteins used, and other secreted proteins not specifically mentioned above may also be used, since it is known that the oocyte and early embryo have mRNA encoding for many different secreted proteins.

### EXAMPLE

### Example 1

### Characterization of Secreted Proteins in Oocyte Spent Medium

Oocyte maturation takes place in an environment maintained by bidirectional interactions of the gamete with the surrounding somatic cells. Oocytes control this environment by releasing factors that act on cumulus and mural granulosa cells ([9], [10]) but the somatic cell signals affecting the oocyte are less well understood. Although the effects of EGF on oocyte maturation and cumulus expansion have long been established ([11], [12]), the physiological relevance of the EGF network during ovulation has only recently come into focus. Biochemical, genetic and pharmacological evidence now support the concept that LH action during ovulation is mediated by secretion of EGF-like factors ([13], [14], [15], [16]). EGF-like growth factor expression is dramatically increased prior to ovulation and these growth factors mimic many of the LH effects on oocyte maturation and cumulus expansion in vitro [14]. The presence of these growth factors in the preovulatory follicle is not restricted to rodents but also found in other species ([17], [18]). In humans, Amphiregulin (Areg) and Epiregulin (Ereg) mRNA have also been detected in granulosa cells [19] and found in the follicular fluid (FF) after hCG stimulation [20]. More importantly, decreased accumulation of AREG in the FF was observed in follicles yielding an immature oocyte [20]. Thus, the induction of these factors plays a critical role at the time of ovulation in all species thus far investigated. It should be noted that in mice, several reports have indicated secretion of EGF-like growth factors by the decidua, implying that embryos are exposed to these growth factors also during implantation ([21], [22]).

In vitro exposure of cumulus oocyte complexes to EGF improves the progression of the oocyte through MI and MII as well as promoting the competence for pre-implantation embryo development. This is true for rodents ([23], [24], [25]), as well as for numerous other species. Exposure of bovine oocytes to EGF during maturation permits the development to blastocysts in a chemically-defined medium, and, importantly, promotes protein synthesis ([26], [27]). In zebrafish, EGF and TGFα promote oocyte maturation, possibly by regulation of activin [28]. We used mice in which Areg is knocked out to evaluated oocyte competence. Our preliminary studies in the Areg-null mice indicated that these growth factors are important for oocyte translation and to establish developmental competence of the oocyte. The AREG measurement in follicular fluid from women undergoing IVF further supports this concept [20].

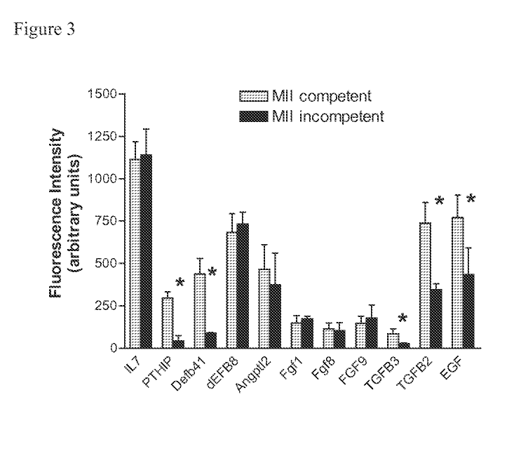

We characterized of the patterns of maternal mRNA translation during oocyte maturation [29]. The translation dataset was then analyzed using Panther GO and searched for signal peptides using Ensembl or scanned manually. A significant number of transcripts translated in the oocytes code for secreted proteins. We compared proteins in two groups of oocytes at the MII stage of development (these are oocytes ready to be fertilized and are the stage retrieved during IVF). Competent oocytes were from wild type mice, whereas the incompetent oocytes were from Areg knockout mice. These mice have reduced fertility and about 50% of the oocytes do not fertilize when tested in an in vitro fertilization protocol (IVF protocol). The results (FIG. 3) showed that incompetent oocytes secreted very little PTHPIP, Defb41, TGFB3, and about 50% less TGFB2 and EGF (see asterisk). FGF8 and FGF1 levels were similar in the two oocytes groups. These results show that the amount of protein can be used as a signature for oocyte developmental competence. For example, decreased secretion of PTHPIP, Defb41, TGFβ3, TGFβ2, and EGF can be used as a signature for an oocyte with compromised developmental competence.

### Example 2

### Validation of the Secretion Patterns by ELISA

To confirm that the pattern of translation reflects secretion of the encoded protein, we tested IL-7 secretion by cultured oocytes. IL-7 mRNA is detected in rat oocytes, and is significantly decreased in human follicular fluid of patients who failed IVF ([30], [31]). We showed that IL-7 mRNA was recruited to the polysomes during mouse oocyte maturation (6.5 fold increase, P<0.01). IL-7 was detected by ELISA (B&D) in the spent medium in a pool of 40-50 of mouse oocytes matured to MII in vitro (˜10-20 pg/50 μl). On average, denuded oocytes released 8.3±2 fg IL-7/oocyte. Confirming the translation data, IL7 secretion by MII oocytes was 5-fold higher than oocytes maintained in GV. IL-7 secretion was also detected in oocytes enclosed by cumulus cells. Thus, these data validated that recruitment to the polysomes is associated with increased release of secretory products.

### Example 3

### Validation of DNA Barcode Assay on Human Cumulus Oocytes

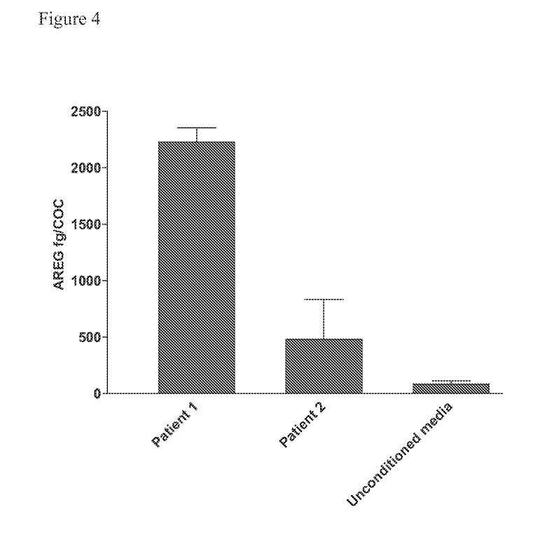

This example illustrates the use of the present invention with human biological in vitro fertilization conditioned media specimens. We employed a DNA barcode assay to measure secreted proteins from media incubated with human cumulus oocytes complexes from patients undergoing in vitro fertilization treatment. Human cumulus oocyte complexes were incubated with standard IVF media in a volume of 500 microliters for a period of 4-6 hours immediately after oocyte retrieval. The conditioned media was then analyzed using the DNA barcode assay protocol as described above. The DNA oligomer was functionalized by a 5′ attachment of an amino-modified group and then conjugated to an antibody recognizing AREG (R&D Systems, Inc.) using a commercially available kit (Solulink Cat #A-9202-001). A second antibody recognizing a different AREG epitope (R&D Systems, Inc) was conjugated to magnetic beads using a commercially available coupling kit (Invitrogen Cat #14311D). The assay was optimized for amount of antibody preparations and type of tubes (DNA LoBind tubes, Eppendorf) using a recombinant purified AREG protein (R&D Systems, Inc.) The conditioned media was mixed and incubated with the antibody linked to the DNA oligomer for 2 hours. The magnetic beads conjugated to the other antibody were then added and the mixture mixed by rotation for an additional hour. The complexes were then isolated by magnetic separation and washed extensively to remove the residual unbound antibody-DNA oligomer. The samples were then resuspended in ultrapure water and the complexes dissociated. The supernatant was then quantitated by qPCR using standard conditions. The concentration of AREG was determined by interpolation on a standard curve, with the standard curve being generated by simultaneously assaying samples with known amounts of recombinant AREG protein. The data in FIG. 4 demonstrate that methods of the present invention detected secreted proteins at levels sensitive enough to measure single cumulus-oocyte complexes.

### REFERENCES

1. Fechner, A. J. and P. G. McGovern, The state of the art of in vitro fertilization. Front Biosci (Elite Ed), 2011. 3: p. 264-78.
2. Albertini, D. F., et al., Nuclear and cytoplasmic changes during oocyte maturation. 1991, Springler-Verlag, New York: Bavister BD in Preimplantation embryo development.
3. Gosden, R. and B. Lee. Portrait of an oocyte: our obscure origin. J Clin invest, 2010. 120(4): p. 973-83.
4. Combelles, C. M. H., et al., Assessment of nuclear and cytoplasmic maturation in in-vitro matured human oocytes Hum. Reprod., 2002. 17(4): p. 1006-1016.
5. Gandolfi, T. A. and F. Gandolfi, The maternal legacy to the embryo: cytoplasmic components and their effects on early development. Theriogenology, 2001. 55(6): p. 1255-76.
6. Eppig, J. J., Coordination of nuclear and cytoplasmic oocyte maturation in eutherian mammals. Reprod Feral Dev, 1996. 8: p. 485-489.
7. DasGupta, R., et al., Functional genomic analysis of the Wnt-wingless signaling pathway. Science, 2005. 308(5723): p. 826-33.
8. Osterfeld, S. J., et al. Multiplex protein assays based on real-time magnetic nanotag sensing. Proc Natl Acad Sci USA, 2008. 105(52): p. 20637-40.
9. Su, Y. Q., K. Sugiura, and J. J. Eppig, Mouse oocyte control of granulosa cell development and function: paracrine regulation of cumulus cell metabolism. Semin Reprod Med, 2009. 27(1): p. 32-42.
10. Matzuk, M. M., et al., Intercellular communication in the mammalian ovary: oocytes carry the conversation. Science, 2002. 296(5576): p. 2178-80.
11. Downs, S. M., Specificity of epidermal growth factor action on maturation of the murine oocyte and cumulus oophorus in vitro. Biol Reprod, 1989. 41(2): p. 371-9.
12. Dekel, N. and I. Sherizly, Epidermal growth factor induces maturation of rat follicle-enclosed oocytes. Endocrinology, 1985. 116(1): p. 406-9.
13. Conti, M., et al., Role of the epidermal growth factor network in ovarian follicles. Mol Endocrinol, 2006. 20(4): p. 715-23.
14. Park, J. Y., et al., EGF-like growth factors as mediators of LH action in the ovulatory follicle. Science, 2004. 303(5658): p. 682-4.
15. Ashkenazi, H., et al., EGF-like growth factors: endogenous mediators of the ovulatory response? 2004. (submitted).
16. Hsieh, M., et al., Luteinizing hormone-dependent activation of the epidermal growth factor network is essential for ovulation. Mol Cell Biol, 2007. 27(5): p. 1914-24.
17. Lindbloom, S. M., et al., Potential involvement of EGF-like growth factors and phosphodiesterases in initiation of equine oocyte maturation. Animal Reproduction Science. In Press, Corrected Proof.
18. Robert, C., et al., Subtractive hybridization used to identify mRNA associated with the maturation of bovine oocytes. Molecular Reproduction and Development, 2000. 57(2): p. 167-175.
19. Ben-Ami, I., et al., Novel function of ovarian growth factors: combined studies by DNA microarray, biochemical and physiological approaches. Mol Hum Reprod, 2006. 12(7): p. 413-9.
20. Zamah, A. M., et al., Human oocyte maturation is dependent on LH-stimulated accumulation of the epidermal growth factor-like growth factor, amphiregulin. Hum Reprod, 2010. 25(10): p. 2569-78.
21. Das, S. K., et al., Expression of Betacellulin and Epiregulin Genes in the Mouse Uterus Temporally by the Blastocyst Solely at the Site of its Apposition Is Coincident with the “Window” of Implantation. Developmental Biology, 1997. 190(2): p. 178-190.
22. Wang, H. and S. K. Dey, Roadmap to embryo implantation: clues from mouse models. 2006. 7(3): p. 185-199.
23. De La Fuente, R., M. J. O'Brien, and J. J. Eppig, Epidermal growth factor enhances preimplantation developmental competence of maturing mouse oocytes Hum. Reprod., 1999. 14(12): p. 3060-3068.
24. Smith, S., S. M. Pfeifer, and J. A. Collins, Diagnosis and management of female infertility. JAMA, 2003. 290(13): p. 1767-70.
25. Ben-Yosef, D., et al., Rat oocytes induced to mature by epidermal growth factor are successfully fertilized. Mol Cell Endocrinol, 1992. 88(1-3): p. 135-41.
26. Park, K. W., K. Iga, and K. Niwa, Exposure of bovine oocytes to EGF during maturation allows them to develop to blastocysts in a chemically-defined medium. Theriogenology, 1997, 48(7): p. 1127-35.
27. Goff, A., et al., Protein Synthesis during Maturation of Bovine Oocytes, Effect of Epidermal Growth Factor Reproduction in Domestic Animals, 2001. 36(1): p. 19-24.
28. Pang, Y. and W. Ge, Epidermal Growth Factor and TGF{alpha} Promote Zebrafish Oocyte Maturation in Vitro: Potential Role of the Ovarian Activin Regulatory System Endocrinology, 2002. 143(1): p. 47-54.
29. Chen, J., et al., Genome-wide analysis of translation reveals a critical role for deleted in azoospermia-like (Dazl) at the oocyte-to-zygote transition. Genes Dev, 2011. 25(7): p. 755-66.
30. Cheng, Y., et al., Oocyte-expressed interleukin 7 suppresses granulosa cell apoptosis and promotes oocyte maturation in rats. Biol Reprod, 2011. 84(4): p. 707-14.
31. Ostanin, A. A., et al., Role of cytokines in the regulation of reproductive function. Bull Exp Biol Med, 2007. 143(1): p. 75-9.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications, patent documents, and accession numbers cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1. A method of identifying an oocyte that is suitable for use in in vitro fertilization, the method comprising:
culturing a candidate oocyte in an oocyte culture medium;
obtaining a sample of the culture medium in which the oocyte is cultured;
determining the amount of at least one protein selected from the group consisting of BMP15, IL-7, FGF1, TGFβ2, TGFβ3, PTHPIP, Def41b, BMP5, Gremlin1, Rspo2, EGF, and CSF1 in the culture medium.

2. The method of claim 1, further comprising comparing the amount of the at least one protein to the amount in the spent medium from a competent oocyte.

3. The method of claim 1, wherein the method comprises determining the amounts of at least two, at least three, at least four, at least five, at least six, at least seven, at least eight, at least nine, at least ten, or at least eleven proteins selected from the group consisting of BMP15, IL-7, FGF1, TGFβ2, TGFβ3, PTHPIP, Def41b, BMP5, Gremlin1, Rspo2, EGF, and CSF1 in the oocyte culture medium.

4. The method of claim 3, further comprising comparing the amounts of the at least two, at least three, at least four, at least five, at least six, at least seven, at least eight, at least nine, at least ten, or at least eleven proteins to the amounts in the spent medium from a competent oocyte.

5. The method of claim 3, wherein the method comprises determining the amount of IL-7 in the oocyte culture medium.

6. The method of claim 1, wherein the method comprises determining the amounts of BMP15, IL-7, FGF1, TGFβ2, TGFβ3, PTHPIP, Def41b, BMP5, Gremlin1, Rspo2, EGF, and CSF1 in the oocyte culture medium

7. The method of claim 1, wherein the method comprises determining the amounts of at least two, three, four, five, or six proteins selected from the group consisting of IL-7, TGFβ2, TGFβ3, EGF, PTHPIP, CSF1, and Defb41 in the oocyte culture medium.

8. The method of claim 7, wherein the method comprises determining the amount of IL-7, TGFβ2, TGFβ3, EGF, PTHPIP, CSF1, and Defb41 in the oocytes culture medium.

9. The method of claim 7, wherein the method comprises determining the amounts of IL-7, TGFβ2, EGF, PTHPIP, and CSF1 in the oocyte culture medium.

10. The method of claim 7, wherein the method comprises determining the amount of IL-7, EGF, PTHPIP, and CSF1 in the oocyte culture medium.

11. The method of claim 1, wherein the method comprises determining the amounts of IL-7, TGFβ2, TGFβ3, PTHPIP, Def41b, BMP5, Rspo2, EGF, and CSF1 in the culture medium.

12. The method of claim 7, further comprising determining the amount of at least one protein selected from the group consisting of Gremlin1, BMP15, and FGF1 in the oocyte culture medium.

13. The method of claim 7, further comprising determining the amount of at least two proteins selected from the group consisting of Gremlin1, BMP15, and FGF1 in the oocyte culture medium.

14. The method of claim 1, wherein the oocyte is fertilized.

15. A kit comprising reagents to determine the amount of two or more proteins selected from the group consisting of BMP15, IL-7, FGF1, TGFβ2, TGFβ3, PTHPIP, Def41b, BMP5, Gremlin1, Rspo2, EGF, and CSF1 in an oocyte culture medium.

16. The kit of claim 15, comprising reagents to detect the amount at least three, at least four, at least five, at least six, at least seven, at least eight, at least nine, at least ten, or at least eleven proteins selected from the group consisting of BMP15, IL-7, FGF1, TGFβ2, TGFβ3, PTHPIP, Def41b, BMP5, Gremlin1, Rspo2, EGF, and CSF1 in the oocyte culture medium.

17. The kit of claim 15, comprising reagents to detect the amounts of BMP15, IL-7, FGF1, TGFβ2, TGFβ3, PTHPIP, Def41b, BMP5, Gremlin1, Rspo2, EGF, and CSF1 in the oocyte culture medium.

18. The kit of claim 15, comprising reagents to detect the amounts of at least two, three, four, five, or six proteins selected from the group consisting of IL-7, TGFβ2, TGFβ3, EGF, PTHPIP, CSF1, and Defb41 in the oocyte culture medium.

19. The kit of claim 15, comprising reagents to detect the amounts of IL-7, TGFβ2, TGFβ3, EGF, PTHPIP, CSF1, and Defb41 in the oocyte culture medium.

20. The kit of claim 15, comprising reagents to detect the amounts of IL-7, TGFβ2, EGF, PTHPIP, and CSF1 in the oocyte culture medium.

21. The kit of claim 15, comprising reagents to detect the amounts of IL-7, EGF, PTHPIP, and CSF1 in the oocyte culture medium.

22. The kit of claim 15, comprising reagents to detect the amounts of IL-7, TGFβ2, TGFβ3, PTHPIP, Def41b, BMP5, Rspo2, CSF1, and EGF in the oocyte culture medium.

23. The kit of claim 18, further comprising reagents to detect the amounts of at least one protein selected from the group consisting of Gremlin1, BMP15, and FGF1 in the oocyte culture medium.

24. The kit of claim 18, further comprising reagents to detect the amounts of at least two proteins selected from the group consisting of Gremlin1, BMP15, and FGF1 in the oocyte culture medium.

25. The kit of claim 15, wherein the kit comprises reagents for a DNA barcode assay.
